(19) United States
(12) Patent Application Publication
(10) Pub. No.: US 2013/0304030 A1
(43) Pub. Date: Nov. 14, 2013

(54) MEDICAL GUIDEWIRE SYSTEM WITH PLURAL PARALLEL GUIDEWIRES

(71) Applicants: William A. Gray, New York, NY (US); Edward I. Wulfman, Woodinville, WA (US)

(72) Inventors: William A. Gray, New York, NY (US); Edward I. Wulfman, Woodinville, WA (US)

(73) Assignee: Vadiswire Corp., Woodinville, WA (US)

(21) Appl. No.: 13/668,126

(22) Filed: Nov. 2, 2012

Related U.S. Application Data
(60) Provisional application No. 61/556,181, filed on Nov. 5, 2011.

(51) Int. Cl. A61M 25/09

(52) U.S. Cl. CPC A61M 25/09
USPC 604/510; 604/528

(57) ABSTRACT

Guidewire system includes a guidewire jacket, at least two guidewires, and a handle. The guidewires are initially constraint within the guidewire jacket and may be introduced into a diagnostic or other catheter to deliver the guidewires to a target region in a patient's vasculature. The handle detachably retains the guidewires so that they may be advanced simultaneously with the guidewire jacket. The guidewires may be then detached and manipulated individually to locate them properly within the region. After removing the guidewire jacket and the diagnostic catheter, the two or more guidewires provide a trackable yet flexible path for the introduction of interventional catheter or sheath.

### CROSS REFERENCE TO RELATED TO APPLICATIONS

This application claims the benefit of U.S. Provisional Application No. 61/556,181 (Attorney Docket No. 42807-703.101), filed Nov. 5, 2011, the entire contents of which are incorporated herein by reference.

### BACKGROUND OF THE INVENTION

1. Field of the Invention

The technical field of the present invention relates to medical guidewires and access systems.

Medical guidewires are typically used to access areas of interest in the body for diagnostic, interventional and surgical procedures and the like. Guidewires are usually the first used first to access a site of interest in a body lumen and then as a rail to slide diagnostic and interventional catheters and devices to the site. Various guidewire sizes have been produced for different anatomical and procedural situations. Typically, guidewires have a stiff proximal section to enhance axial pushability and rotational torque and a flexible distal portion to accommodate tortuosity. Guidewires typically have a floppy tip or other soft structure at the distal end to prevent trauma.

Because there are so many anatomical and procedural situations, there are large number of guidewire designs with diameters typically ranging from 0.010 inch to 0.038 inch and lengths ranging from 90 cm to 300 cm or more, with different grind profiles, coatings, and radiopaque tips. These parameters are well known to those familiar in the art.

Some recent efforts have been made to design guidewires with variable parameters than can be changed during a procedure to adjust to fit anatomical and procedural situations. More specifically guidewires have been designed to provide a varying level of stiffness or flexibility at certain sections of the guidewire. These adjustable guidewires are typically constructed of coaxial guidewires and tubes, with a guidewire being moved inside a tube to provide different flexibilities. However, these adjustable guidewires are limited by high friction between internal guidewire and tube, as well as how much adjustment can be accomplished.

One particular challenge for conventional guidewires is the introduction of a diagnostic catheter into the common carotid artery from the aortic arch, which challenge can be exacerbated by disease, and it's common to use a very stiff/supportive guidewire, such as a 0.35 inch guidewire. Guidewire placement cannot usually be achieved in these situations because the tortuosity will limit wire advancement and/or the wire will displace the diagnostic catheter from its position in the difficult anatomy. Therefore, the procedure will either become prolonged due to repeated attempts at access (exposing the patient to increasing risk of complication), or cannot proceed due to a failure of placement and a lack of subsequent access with an interventional guide or sheath.

To overcome this difficulty, Cardaioli et. al. (2009) J. Endovasc. Ther. 16:649-651, have proposed placing multiple 0.035 access guidewires from the aortic arch to the internal carotid artery in order to place a large guiding catheter that can accommodate several 0.035 wires. This approach, however, requires placing a larger introducer sheath which cannot be done in every patient, and interventionalists generally prefer to use smaller catheters until access is guaranteed because of risks of larger catheters.

Another challenging use for conventional guidewires is the interventional treatment of mesenteric pathology, e.g. oncologic, aneurysmal, atherosclerotic/obstructive, etc. For example, the placement of stent-grafts (generally large and stiff devices) in a highly tortuous splenic artery can be very difficult. Use of a stiff guidewire, such as a 0.035 inch guidewire, can straighten the artery making placement of the stent-graft difficult due to the creation of a pseudo-stenosis and loss of both anatomic landmarks and normal flow.

Another challenge arises from the numerous separate, time consuming, steps that can prolong the placement of stent-grafts in abdominal aortic aneurysms. Specifically, placement of a contralateral leg of such stent-grafts can require many steps and multiple guidewire exchanges in order to confirm intra-graft position, establish a marker system for required angiographic distance assessment, and then to locate a very stiff guidewire to allow advancement of the catheter curing the contralateral stent-graft.

A further challenge for conventional guidewires is the placement of an access guidewire into the iliac and femoral arteries from the contralateral femoral artery via the aortic bifurcation when the birfurcation is acute, calcified, or of a difficult geometry.

Another difficult challenge for interventionalists and surgeons is the access of a contralateral gate of a bifurcated stent graft for treatment of abdominal aortic aneurysms. During these procedures it is often necessary to place a guidewire in the contralateral gate, and verify that it is properly inside via two dimensional x-ray imaging.

Yet another challenge for interventionalists is the inability to inject contrast media for fluoroscopic (x-ray) visualization during placement of a 0.035 guidewire via a small 5-6 French diagnostic or guiding catheter. The small lumens of these catheters accommodate only the guidewire which substantially limits introduction of contrast media for distal visualization.

For these reasons, it would be desirable to provide a highly adjustable, easy and intuitive to use, low friction guidewire system that can accommodate almost any anatomical challenge. In particular, it would be desirable to provide guidewires and guidewire systems which can provide excellent trackability and necessary support while minimizing the stiffness of the guidewires or guidewire system to limit straightening of the target anatomy. It would be further desirable that the guidewires and guidewire systems could be used in a variety of anatomies, including at least for access to carotid arteries, for placement of stent-grafts in aneurysms, including both splenic aneurysms and abdominal aortic aneurysms, and elsewhere. It would be still further desirable if the deployment of the guidewires and guidewire systems were simplified to reduce the number of steps required for placement prior to introduction of the desired interventional or other catheter. It would be still further desirable if a guidewire system allowed contrast media to be injected through a standard 5-6 French diagnostic or guiding catheter during placement of wires in difficult anatomy. At least some of these objectives will be met by the inventions described below.

2. Description of Background Art

Cardaioli et al. (2009) J. Endovasc. Ther. 16: 649-651 has been described above. A guidewire control station used for managing multiple guidewires for bifurcated stent delivery is described in U.S. Pat. No. 7,645,273 and U.S. 2006/0074484. Variable stiffness guidewires are described in U.S. Pat. No. 7,402,141 and U.S. 2010/0305475.

### SUMMARY OF THE INVENTION

According to the present invention, a medical guidewire system comprises a plurality of parallel relatively small guidewires which can be individually manipulated for wire deployment and, after wire deployment, utilized as one combined, larger guidewire for placement of an interventional or therapeutic catheter. By “relatively small,” it is meant that the individual wires are of a lesser caliber than the nominal internal diameter catheter in which they are intended to be used. The guidewire systems of the present invention will typically be placed through a pre-placed diagnostic or other catheter or sheath which is located at a target region in a patient's vasculature. Although exemplified by use in the vasculature, the systems and methods of the present invention could also find use in other body lumens, such as in the urinary tract. A plurality of individual guidewires, each of which will usually have a diameter below 0.031 inch, usually below 0.025 inch, more usually below 0.018 inch, frequently being 0.014 inch or smaller, are initially introduced to the diagnostic or other catheter using a guidewire jacket which is typically a tubular body which surrounds and constrains the multiple guidewires being introduced. The guidewire jacket is first introduced into the diagnostic or the catheter, and the multiple guidewire then advanced through the jacket and through the diagnostic catheter until they reach a location near a distal end of the diagnostic catheter. Then, individual ones of the multiple guidewires may be advanced beyond the diagnostic catheter to the target region in the vasculature until two, three, four, or more individual guidewires have all been located at the target region. After the multiple guidewires are properly located, the jacket and/or diagnostic catheter may be removed over the guidewires, leaving the multiple guidewires in place and available for use as a combined or assembled guidewire system. A desired interventional catheter, sheath, or other interventional tool may then be advanced over the combined guidewire assembly, and a subsequent treatment, intervention, or diagnosis at the target region may be performed using the guidewire, catheters, sheaths, and/or tools. The use of multiple, small diameter, guidewires has been found to provide both trackability and support equivalent to those achieved with larger guidewires while presenting a much less stiff structure in regions of tortuosity and facilitating placement. The multiple guidewire assemblies will often also occupy less cross-section while in the jacket or diagnostic catheter, precipitating the introduction of contrast or other media through the jacket and/or diagnostic catheter.

In a first specific aspect of the present invention, a guidewire system comprises a guidewire jacket, at least two guidewires, and optionally a handle. The guidewire jacket has a proximal end, distal end, and a connector hub at its proximal end. The at least two guidewires are slidably received in a lumen of the guidewire jacket, and each guidewire has a length which is greater than twice that of the guidewire jacket. The optional handle removeably receives each guidewire and may include a distal connector which detachably attaches to the connector hub on the guidewire jacket.

As described in greater detail below in connection with the methods of the present invention, the connector hub on the guidewire jacket may be attached to a proximal end of a pre-placed diagnostic or other catheter after the guidewire jacket has been fully inserted into the pre-placed catheter. The handle may then be used to simultaneously advance the at least two guidewires fully into the guidewire jacket and the diagnostic catheter, and the distal connector on the handle may then be attached to the connector hub on the jacket, thus allowing individual guidewires to be removed from the handle and manipulated while the other guidewires remain attached to the handle and immobilized relative to the guidewire jacket, diagnostic catheter, and vasculature. Such a construction greatly simplifies the placement and use of the individual guidewires making placement of multiple guidewires manageable by even less experienced physicians.

In particular embodiments of the guidewire systems of the present invention, a distal region of the guidewire jacket will consist of a tubular element which is free from exterior structure, such as balloons. The distal region of course may include other integrated features, such as radiopaque markers and alike. In other specific embodiments, the proximal connector on the guidewire jacket will comprise a luer and the distal connector on the handle will comprise a Touhy Borst valve. A removable retainer will typically be provided to hold a distal region of each guidewire to the connector hub on the guidewire jacket so that the guidewires remain fixed relative to the guidewire jacket while the jacket is being advanced into the diagnostic catheter.

In a still further specific embodiment of the guidewire system of the present invention, the system will be assembled for use with the distal tips of the guidewires received in a proximal end of the guidewire jacket lumen. The removable retainer will also be in place holding the guidewires, and the handle will be positioned proximally of the retainer and the connector hub so that the guidewire jacket may be placed into the diagnostic or other catheter, the connector hub attached to a hub on a catheter, and the retainer remove. The handle may then be advanced to introduce the guidewires through the guidewire jacket lumen. Such pre-assembled guidewire systems may be maintained in sterilized condition within a suitable medical device container, such as a box, tray, bag, or the alike.

The individual guidewires of the guidewire system of the present invention will usually be relatively flexible, typically having diameters in the ranges set forth above, and in some cases from 0.005 inch to 0.030 inc. Usually, however, at least one of the guidewires will have a stiffness which is greater than that of at least one other guidewire. In an exemplary systems, there will be one relatively stiff guidewire, and two relatively flexible guidewires in a single system, although other systems may comprise at least one relatively flexible guidewire and two relatively stiff guidewires. The guidewires may further comprise removable torquers attached to each guidewire. The systems may still further comprise a guidewire loop which receives proximal ends of the guidewires which extend proximally from the handle to assist in management. In still further specific embodiments, the guidewire system will comprise at least one larger guidewire having a distal diameter in the range from 0.014 inch to 0.028 inch and at least one smaller guidewire having a distal diameter in the range from 0.009 inch to 0.018 inch. Such system may consist of the one larger guidewire and two smaller guidewires and no additional guidewires or may consist of the one smaller guidewire and two larger guidewires and no additional guidewires. In other systems, all guidewires may be of the same diameter and ther may be from two to five total guidewires.

In a further specific embodiment of the guidewire system of the present invention, the system will consist of one larger diameter guidewire and one smaller diameter guidewire having pigtail distal end, usually being pre-shaped. At least one of these two guidewires will also usually have radiopaque markers at or near their distal end(s). Such guidewire systems are particularly suitable for assisting in placement of stent-grafts in abdominal aortic aneurysms.

In a second specific aspect of the present invention, methods for advancing catheter through a patient's vasculature or other body lumens are provided. A guidewire jacket is introduced into a lumen of a first catheter which was pre-placed into the vasculature to reach a target region. A first guidewire is advanced from the guidewire jacket into and through the first catheter lumen to the target region. A second guidewire also advanced from the guidewire jacket into and through the first catheter lumen to the target region in parallel to the first guidewire. Optionally, third, fourth, fifth, and additional guidewires could also be advanced, although usually no more than four guidewires would be advanced in total. After the guidewires are advanced and located, the first catheter and optionally the guidewire jacket are removed, leaving the guidewires in place. The jacket may optionally be left in place over the guidewires, but it will be eventually be necessary to remove any proximal hub or other structure from the jacket if the jacket is to remain in place when a interventional, therapeutic, or other catheter is advanced over the guidewire/jacket assembly. A second catheter may then be advanced over the parallel guidewires (and optionally jacket) to the target region. As discussed above in connection with the system of the present invention, the use of multiple parallel guidewires can provide excellent support and trackability while minimizing guidewire stiffness and limiting the risk of guidewire displacement as a second catheter is advanced.

In specific embodiments, introducing the guidewire jacket may comprise connecting a proximal end of the guidewire jacket to proximal end of the first catheter. Distal regions of the guidewires are detachably attached to the proximal end the guidewire jacket, and the guidewires may be detached from the proximal end of the guidewire jacket after the guidewire jacket has been introduced into the lumen of the first catheter but prior to advancing the individual guidewires to the target region.

In further specific aspects of the methods herein, a middle section of each guidewire maybe detachably attached to a handle which allows the guidewires to be simultaneously advance through the guidewire jacket by, in turn, advancing the handle relative to the guidewire jacket. The middle sections of the guidewires to which the handles detachably attached are usually located proximally of the distal end of each guidewire at a distance which positions the distal ends of the guidewires near the distal end of the guidewire jacket when the handle is fully advanced. After the handle has been fully advanced, individual guidewires are detached from the handle, and the detached guidewire manipulated while the other guidewire(s) remain attached to the handle.

Use of a Touhy Borst valve on the handle allows the delivery of contrast medium through the guidewire jacket through the target region when the handles attached to the guidewire jacket. Because the assembly of two or more small wires creates cross-sectional spaces inside a catheter lumen, these spaces allow for introduction of contrast media fluid.

In a still further specific embodiment, the first catheter comprises a diagnostic catheter placed from an aortic arch to a carotid artery. The second catheter delivers a stent to the carotid artery to treat the carotid artery disease.

In a still further specific embodiment, the first catheter comprises a diagnostic catheter disposed in a splenic artery and the target region is an aneurysm. The second catheter is used to deliver a stent-graft to the aneurysm in the splenic artery.

In a still further embodiment of the present invention, the target region comprises a contralateral gate in a stent-graft pre-placed in an aneurysms in the abdominal aortic. A distal end of the first catheter may be placed near but not through the contralateral gate, and the first guidewire is advanced through the gate. First catheter is then advanced over the first guidewire through the gate and into the stent-graft. The first and second guidewires are advanced from the jacket, through the stent-graft so that markers present on the guidewires are visible from the stent-graft contralateral gate to the associated iliac artery. The first catheter and guidewire jacket may then be removed from over the first and second guidewires. The second catheter then typically delivers a contralateral stent-graft to the contralateral gate of the main stent-graft body.

### BRIEF DESCRIPTION OF THE DRAWINGS

FIG. 1 shows a longitudinal cross-sectional view of one example of the invention with several parallel guidewires inside a tubular guidewire jacket.

FIG. 2 shows a cross-sectional view of a similar embodiment of the invention with several parallel guidewires of different geometries inside a tubular guidewire jacket.

FIG. 3 shows a side view of another embodiment of the invention with several parallel guidewires of different geometries.

FIG. 4 shows side view of another embodiment of the invention with several parallel guidewires of different geometries.

FIG. 5 shows a side view of another embodiment of the invention with two guidewires in parallel inside a tubular guidewire jacket.

FIG. 6 shows a side view of another embodiment of the invention with two guidewires of different geometries inside a tubular guidewire jacket.

FIG. 7 shows a side view of another embodiment of the invention with a plurality of guidewires of different geometries inside a tubular guidewire jacket, where one guidewire has an enlarged geometry that is distal to the distal end of the tubular guidewire jacket.

FIG. 8 shows a side view of the same embodiment as FIG. 6, where the one guidewire with an enlarged geometry is pulled into the distal end of the tubular guidewire jacket, thereby causing the guidewires to reversibly jam inside the jacket.

FIG. 9 shows one embodiment of the invention, where the guidewire jacket is removably attached to a proximal guidewire management tool with a luer fitting, and the guidewires are removably held in place by grips in the proximal guidewire management tool.

FIG. 10 shows a similar embodiment of the invention as shown in FIG. 9, with the addition of a peel away introducer placed on the proximal end of the parallel guidewires to assist in inserting the guidewires into the proximal end of a catheter.

FIG. 11 shows an anatomical example of one use of the invention, where the guidewires are inserted through a catheter into the vasculature of a human body and subsequently placed in a carotid artery.

FIG. 12 shows an example of a typical currently available type of access guidewire and the issue of using such a guidewire in challenging anatomy such as highly angulated vasculature.

FIG. 13 shows an example of the invention in a similar anatomy as that shown in FIG. 12, where the benefits of the invention can be seen to accomplish accessing challenging anatomy such as highly angulated vasculature.

FIG. 14 illustrates an exemplary guidewire placement systems constructed in accordance with the principles of the present invention.

FIG. 15 illustrates the connection between a distal end of a guidewire jacket of the system of FIG. 14 with a proximal end of a diagnostic catheter which has been pre-placed at a target region in the patient's vasculature.

FIG. 16 illustrates a handle which is used for management multiple guidewires in the guidewire placement system of FIG. 14.

FIG. 17 illustrates guidewire placement system of FIG. 14 connected to a diagnostic catheter with a first guidewire having been manipulated and advance from a distal end of the diagnostic catheter.

FIGS. 18A through 18E illustrate use of the guidewire placement system of the present invention for advancing a contralateral stent-graft leg in a stent-graft system use for treating an abdominal aortic aneurysm.

FIGS. 19A and 19B illustrate a guidewire placement system of the present invention that includes a guidewire jacket with no handle.

### DETAILED DESCRIPTION

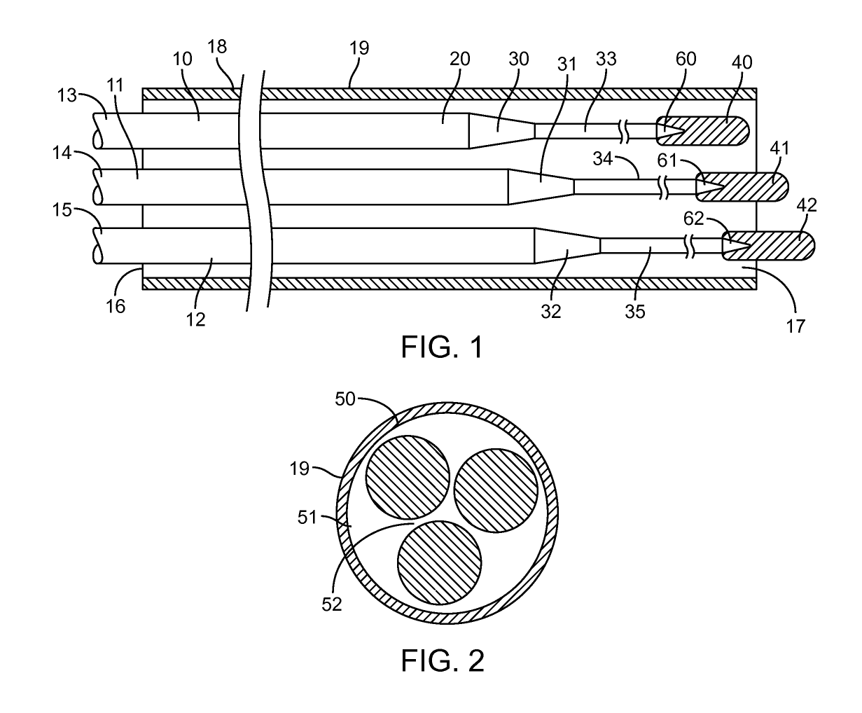

The object of the present invention is to provide an adjustable, easy to use guidewire system that will be able to access most anatomical structures, and provide a capable rail to assist in bringing other catheters, sheaths, and interventional tools to the required sites. In one embodiment, two or more small guidewires of similar geometry are provided side by side inside a tubular jacket, as shown in FIG. 1. These guidewires can be manipulated independently of each other, in a typical fashion well know to those familiar with the art, from a proximal site or manipulated as one assembly. Such a plurality of guidewires in parallel condition provide greater trackability than a single guidewire, yet maintain the flexibility and ability to navigate difficult anatomy as each guidewire is individually advanced. Further, the guidewires can be moved to positions in a staggered or sequential manner in order to provide a transitional zone of flexibility that can be customized during a procedure to meet any situation. Further, because each small guidewire is highly flexible as compared to a single large guidewire, each guidewire can more easily be manipulated into difficult anatomy as compared to a single larger, stiffer guidewire, and yet when several smaller guidewires are placed in the distal anatomy, they can provide significantly more support and trackability than a single guidewire. Further, because each small guidewire is more flexible than a singular larger, stiffer guidewire, each guidewire is likely less traumatic to the body lumen it is being manipulated in. Further, when each guidewire of several parallel guidewires is being manipulated while in a distal anatomy of a body lumen with assistance from a supporting guiding catheter or diagnostic catheter or the like, a smaller guidewire will be less likely to cause the supporting catheter to move out of position while advancing the smaller guidewire, as compared to advancing a larger, stiffer guidewire. Further, several guidewires when placed in distal anatomy have a significant amount of surface area, and thus have a anchoring effect that when combined provide more support for a catheter when traversing the guidewires simultaneously.

Most current state of the art medical guidewires that are used in the beginning of many medical procedures are approximately 1.0 mm in diameter and more specifically 0.035 inch to 0.038 inch in diameter. In addition, most current state of the art guidewires used to transition interventional devices for cardiology and peripheral interventions and the like are smaller, for example 0.018 inch to 0.014 inch in diameter. In contrast, the present invention provides a plurality of smaller guidewires inside a thin flexible tube to match the larger diameter access guidewires. Therefore a parallel and plurality guidewire system can be used first to access the site of interest, and subsequently any guidewire than is no longer required for support well as the outer tube can be removed to leave one smaller guidewire that can be used to deliver interventional devices.

Some parameters that affect the performance of any one guidewire in the system are the diameter of the guidewire at any point in the length of the guidewire and more specifically the proximal section of guidewire; the design of a taper from a larger diameter to a smaller diameter, the starting position of a taper, the length and diameter of a reduced diameter at the smaller end of a taper, the material the guidewire is made from, and lubrication on the guidewire and location thereof. Some parameters that affect the performance of a system of parallel and plural guidewires are the contact area between each guidewire, the contact area of the guidewires to a tube jacket enclosing the guidewires if a jacket is used, lubrication used on the guidewire and the location of such lubrication, the position of features such as tapers relative to each guidewire, and the position of different zones of flexibility relative to each guidewire, the clearance of each guidewire to each other and to the jacket if a jacket is used, the material characteristics of a jacket if a jacket is used. Thus it can be seen that systems can be made of two or more guidewires of different diameters, and each guidewire can also have different profiles as well. Further, these guidewires can be provided in a system at the start of a procedure in an optimum position relative to an enclosing jacket tube for the start of the procedure, and then each guidewire can be manipulated axially for optimum performance of the system depending on the anatomical requirements and the procedural requirements at any point in time. Thus it should be clear that a system of parallel guidewires of even two or three guidewires can provide a very large variation of configurations that can be optimized quickly and easily to meet many anatomical challenges simply by moving the guidewires relative to each other.

A simple example of the present invention includes two or three guidewires of conventional geometry, such as guidewires 10, 11, and 12 shown in FIG. 1, positioned in such a manner that tapers 30, 31, and 32 are in different relative axial positions in order to provide a continuous increase in flexibility towards the distal end. The guidewires can be provided in an initial position for a particular flexibility profile such as shown in FIG. 1, and then each guidewire can be manipulated at the proximal end 13, 15, and 15 to change the flexibility characteristics. In this embodiment, there are three guidewires, two of similar diameter 10 and 11, and one 12 that is larger in the proximal areas 13, 14 and 15. Typically, guidewires are constructed of stainless steel and have profiles ground into them, shown here at tapers 30, 31, and 31, that make the guidewires more flexible in areas where the guidewires would encounter smaller and/or more tortuous body lumens. Further, grinds also make the guidewires less stiff and so less traumatic to these smaller body lumens. At the end of each guidewire is a guidewire tip 40, 41, and 42. These tips are usually made of soft, radiopaque materials such as platinum coils that are welded or soldered to the stainless tips. In the area of the tips, there may be an additional grind 60, 61, and 62 to make the guidewire even more flexible and soft. In many instances, the guidewires have additional material such as plastic or stainless coils to bulk up the area distal over the tapers and the smaller diameters resultant from the grind 33, 34, and 35, in order to make the guidewires a consistent diameter along the full length. For simplicity and clarity, these bulking geometries have not been included in the drawings describing this invention. The guidewires themselves are typically be made from stainless steel, nitinol, and the like. The above guidewire geometries and characteristics are well understood to those familiar to the art. The guidewires used for this invention could be of several diameters, from as small as 0.009 inch up to 0.030 inch at the proximal ends, and more typically from 0.013 inch to 0.022 inch. Lengths of guidewire could be from 90 cm to 300 cm depending on the application. Tapers could be from 1 cm to 30 cm in length, and could be of varying geometries. These tapers are usually made using a centerless grinder. The start of a taper would typically be from 10 cm to 30 cm proximal to the distal tip, but could be longer or shorter depending on the application. In the specific case of this invention, the plurality of parallel guidewires can be provided in the tubular jacket 18 for the purpose of ease of insertion into a catheter, ease of handling in general and to initially mimic a singular guidewire during use. Ideally, the tube jacket would have a very thin and precise wall thickness, in the order of 0.001 inch to 0.004 inch, in order to provide the maximum internal lumen for the parallel guidewires to move in. Examples of exemplary biocompatible materials that meet this requirement are polyimide or PET tubing, and other precision plastics, as well as metal tubing such as stainless steel or nitinol. Outer diameters of a tube jacket would typically be from 0.018 inch to 0.062 inch, and more typically from 0.030 inch to 0.040 inch. Tubes that have been tested for prototypes have been made from polyimide material of 0.001 inch-0.002 inch wall and 0.033 inch to 0.038 inch diameters, with lengths of 50 to 60 cm.

A further advantage of the invention is that each guidewire has very low contact area relative to each other, as compared to a coaxial guidewire system such as a guidewire in a tube. This low contact area means that there is very low friction between each guidewire. In FIG. 2, a plurality of guidewires are shown inside a tubular jacket 19; the contact area 50 between any guidewire and the jacket is very small, and also the contact area 52 between any two guidewires is also very small. This is compared to a larger guidewire inside a tubular jacket where the fit of a larger guidewire and a jacket are close, thus making the contact area and resulting friction higher. This is particularly true when the system is in curves, which would be typical in vascular anatomy, and so the engagement of the inner guidewire or guidewires is forced against the outer tubular jacket. Thus an advantage of this embodiment of the invention is that each guidewire can easily be moved radially and axially, so each guidewire can be steered and advanced to a desired location.

A further reduction in friction between the plurality of guidewires and the tube jacket can be accomplished by coating the guidewires with friction reducing materials such as PTFE, hydrophilic coatings, and the like which are common and well known to those familiar to the art. It may be desirable to coat only certain sections of the guidewire with highly lubricious coatings, and have other sections with minimal or no lubrication. For example, looking again at FIG. 1, it may be useful to only have PTFE coating on the proximal sections of guidewire 13, 14, and 15, as defined as the proximal end to the start of the first tapers 30, 31, and 32, to reduce friction inside of the tube jacket or inside of a catheter. It may be useful to add hydrophilic coatings or the like at certain sections, for instance in zones defined between the tapers 30, 31, and 32, and the tips 40, 41, and 42. Conversely, it may be useful to have no coating in this distal zone to aid in maintaining position in a body lumen while proximal manipulation of the guidewires, such as inserting catheters and the like, is being accomplished.

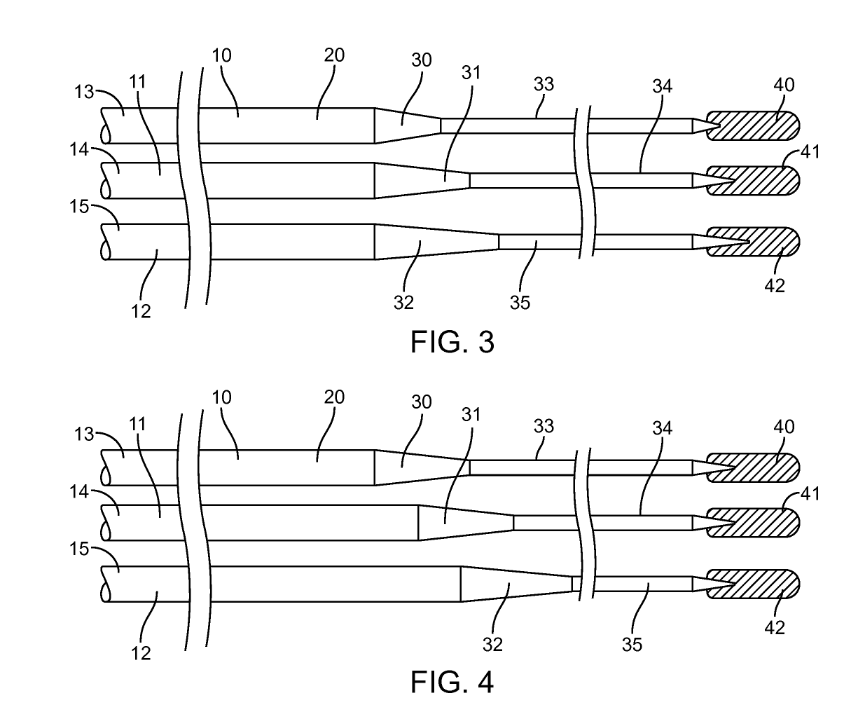

FIG. 3 and FIG. 4 show further embodiments of the invention, where guidewires are provided without being in a tubular jacket. It may be desired for certain reasons to use guidewires without a jacket, such as to provide more clearance between a catheter and guidewires, to completely avoid frictional engagement in tight curves, or to use slightly larger guidewires. In this particular embodiment, each guidewire has unique characteristics; the guidewires are similar in diameter in proximal sections 13, 14, and 15, but each taper 30, 31, and 32 in FIG. 3 are different. This would provide unique transitional flexibilities for each guidewire. In FIG. 4, each guidewire has similar proximal diameters at position 13, 14, 15, and similar tapers 30, 31, and 32, yet each taper starts at a different position relative to the distal tips 40, 41, and 42.

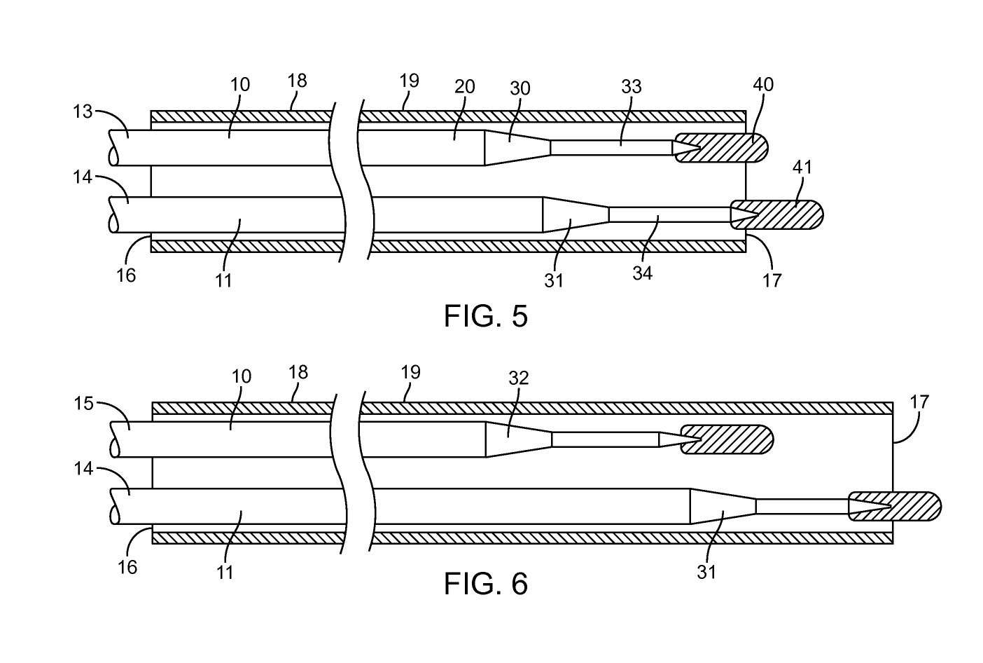

FIG. 5 and FIG. 6 show further embodiments of the invention, where a plurality of guidewires of varying geometry are placed in different positions inside a tubular jacket. In FIG. 5, guidewires of slightly different proximal lengths are positioned such that tapers 30 and 31 overlap, and tips 40 and 41 are at different positions. In FIG. 6, a plurality of guidewires of varying geometry is shown inside a jacket. In this embodiment, the proximal end 15 of guidewire 10 is larger in diameter than that of guidewire 11, and the taper 32 starts at a different position than the taper 31 of guidewire 11. The difference in proximal guidewire diameters would give a different flexibility as compared to two guidewires of similar proximal diameter. Also, having the tapers in different locations would also create different distal flexibilities as compared to that of FIG. 5. Further, it must be understood that each guidewire can be re-positioned axially by manipulating the guidewires from the proximal positions 14 and 15 in FIGS. 5, or 14 and 15 in FIG. 6, to completely customize the flexural characteristics.

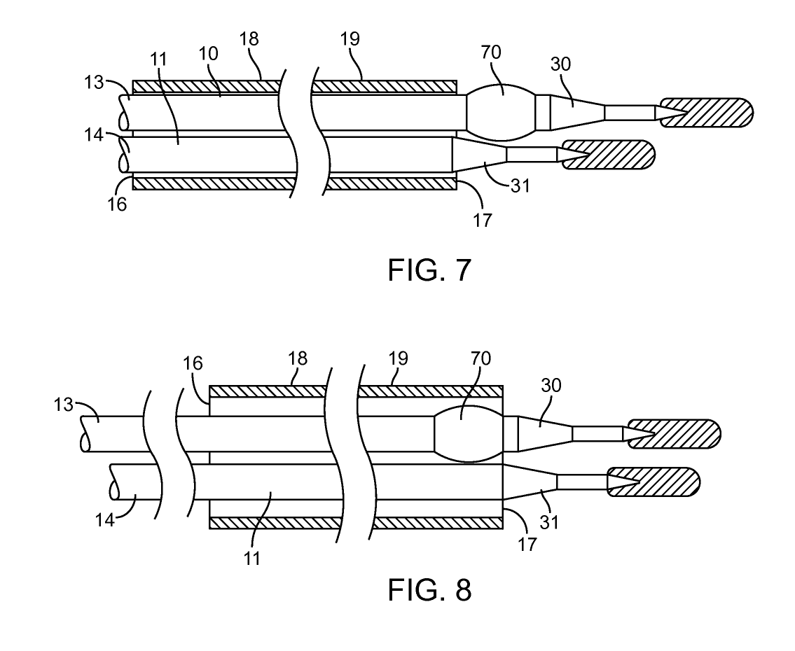

FIG. 7 and FIG. 8 show another embodiment of the invention, where a plurality of parallel guidewires 10 and 11 are inside a tubular jacket 18, where one guidewire has an enlarged geometry 70 such that when the geometry is outside of the jacket mouth 17 as shown in FIG. 7, the guidewires move easily axially and radially. When the guidewire 10 is moved in such a manner that the enlarged geometry 70 is forced inside the distal mouth 17 of jacket 18, the geometry reversibly jams the plurality of guidewires against the inside surface of jacket 18. This feature can be used to reversibly lock the system in place, in order to hold position of the guidewires or to make it easy to manipulate the system as a whole.

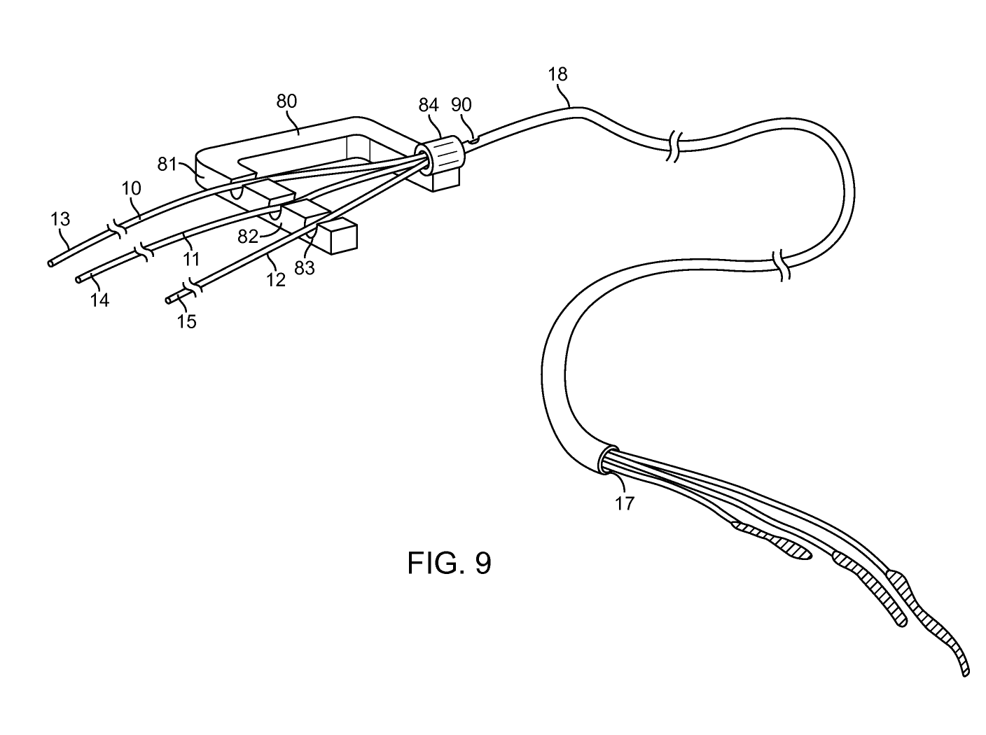

FIG. 9 shows an oblique view of an exemplary embodiment of the invention that includes a proximal guidewire management tool 80 removably attached to the tube jacket 18. The tool 80 can have a luer fitting 84 that can be used to connect the tool to the proximal end of a catheter or other devices. Further, the tool can have a plurality of gripping slots 80, 81, 82 to match the number of guidewires used, and these slots can be arranged on the tool in a manner to spread the guidewires 10, 11, 12 and so make them easy to identify and manipulate. Along these lines, the guidewires themselves can be colored or have identifying marks to clarify each guidewire from one another. Further, radiopaque markers can be placed on the distal ends of the guidewires that would have differentiating characteristics to identify each guidewire while under X-ray fluoroscopy. Examples of these markers are gold or platinum bands of different lengths and numbers particular to each guidewire.

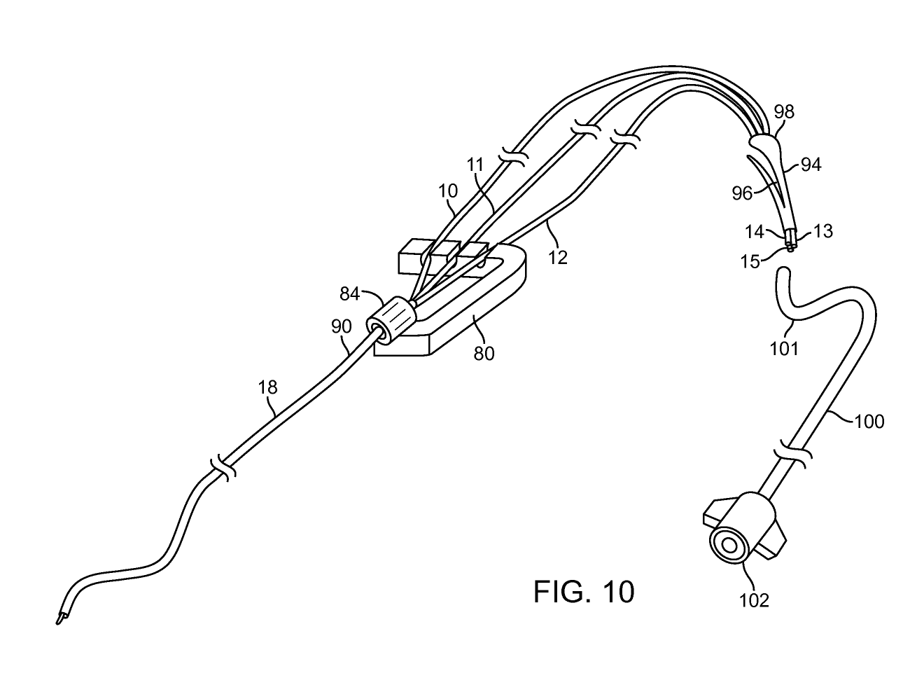

FIG. 10 shows the embodiment of the parallel guidewire management system and guidewires that is described in FIG. 9, but additionally shows a peel away introducer 94 that is added and can be used to corral the distal ends of the plurality of guidewires 13, 14, and 15 by inserting them in the funnel 98 of the introducer. A pre-defined split or cut 96 is provided on the introducer. The introducer is typically made of soft plastic such that when gripped and pulled such that the guidewires are forced into the split, the split is forced to continue to the end of the introducer and it can break away. This would be done after the introducer is used, for example, to insert the distal guidewire ends 13, 14, 15 into the end of a catheter 100. Once a catheter is loaded onto the proximal guidewires, it would be desirable to remove the guidewire management tool 80. Means can be provided to separate the tool from the tube jacket 18, for instance if the jacket is made from a thin plastic such as polyimide, a notch 90 can be provided to weaken the jacket enough that it would break if a twisting force is applied. A thin notch could also be provided in the luer fitting 84 that would provide a clear path for the guidewires to be moved from the center of the luer to the outside. Therefore the management tool 80 could be removed sidewise from the guidewires once the notch 90 is broken. As an alternative, the guidewire management tool and tubing jacket could be removed entirely prior to using the peel away introducer and inserting the distal guidewires into a catheter. Subsequently, the catheter can be inserted over the guidewires until the guidewires exit the catheter proximal end 102, and then advanced with a controlled manner relative to the guidewires into a desired body lumen.

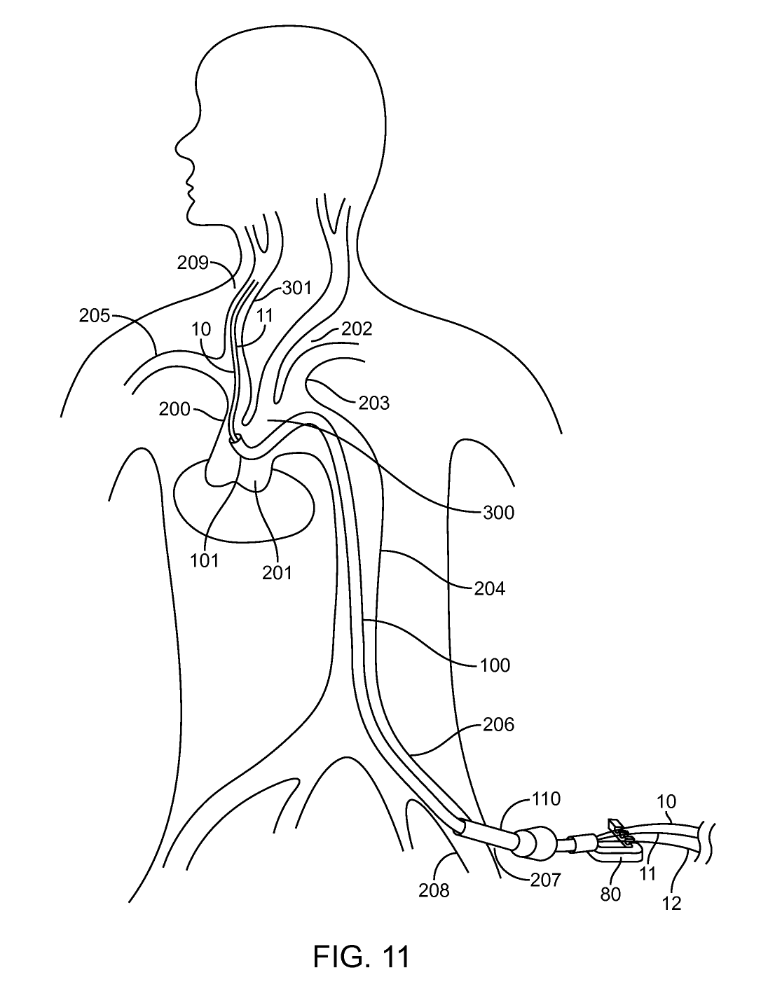

FIG. 11 shows an example of the use of an embodiment of the invention in a clinical scenario where the advantages of the invention would be useful. In this particular presentation, the distal end of parallel guidewires 10 and 11 have been placed in the carotid artery 209. The start of the carotid artery 300 begins, or takes off, on the top of the ascending aorta 201, and in this case creates a difficult angle for a typical access guidewire to traverse. Many older patients needing treatment of their carotid arteries have this type of difficult angle, labeled as a Type III aortic arch. In an article published by Madhwal et al in the Journal of Invasive Cardiology, June 2008, inch Predictors of Difficult Carotid Stenting as Determined by Aortic Arch Angiography inch, Madhwal reports on a population of 48 carotid stenting cases a incidence of 50 percent difficult cases, in which 20.8% had angulated takeoff of the carotid artery, as compared to 4.3% of the population of easy cases had an angulated takeoff. In these difficult cases, average fluoroscopy x-ray time was 58.1 minutes, as compared to 19.1 minutes for easy cases. In a difficult case like this, the method of use of the parallel guidewire system would be that a catheter 100 would be inserted into the vasculature through an access sheath 110 and advanced to the takeoff of the artery 203, and then each guidewire advanced into the desired position 209. Because each guidewire is relatively small, the distal end of the catheter 101 is not moved significantly during guidewire advancement. Once each guidewire is properly positioned, the proximal guidewire management tool 80 is removed, the catheter 100 is removed and an appropriate sheath is advanced over the guidewires and into the carotid artery. Typically this sheath is a larger, stiffer sheath that can accommodate interventional catheters with stents; for example a 6 F Cook Balkan sheath. A difficult situation can be encountered when advancing a very stiff sheath into an angled vasculature as shown, in that the sheath can be too stiff and force a guidewire out of position. This points to an additional advantage of the invention, where several guidewires placed distally in the vessel create enough friction by virtue of total surface area against the vessel, that the guidewires stay in place and the sheath can follow the guidewires into the takeoff of the vessel. Once the sheath is in place, the parallel guidewires can all be removed, or all but one, and then additional guidewires and interventional catheters or the like can be advanced through the sheath for treatment purposes.

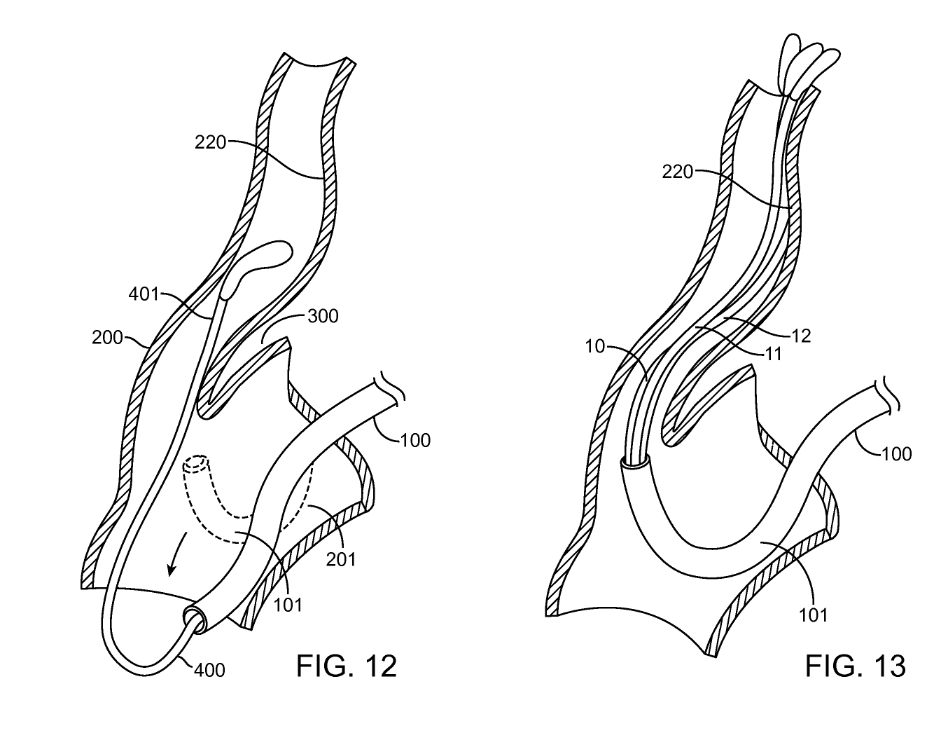

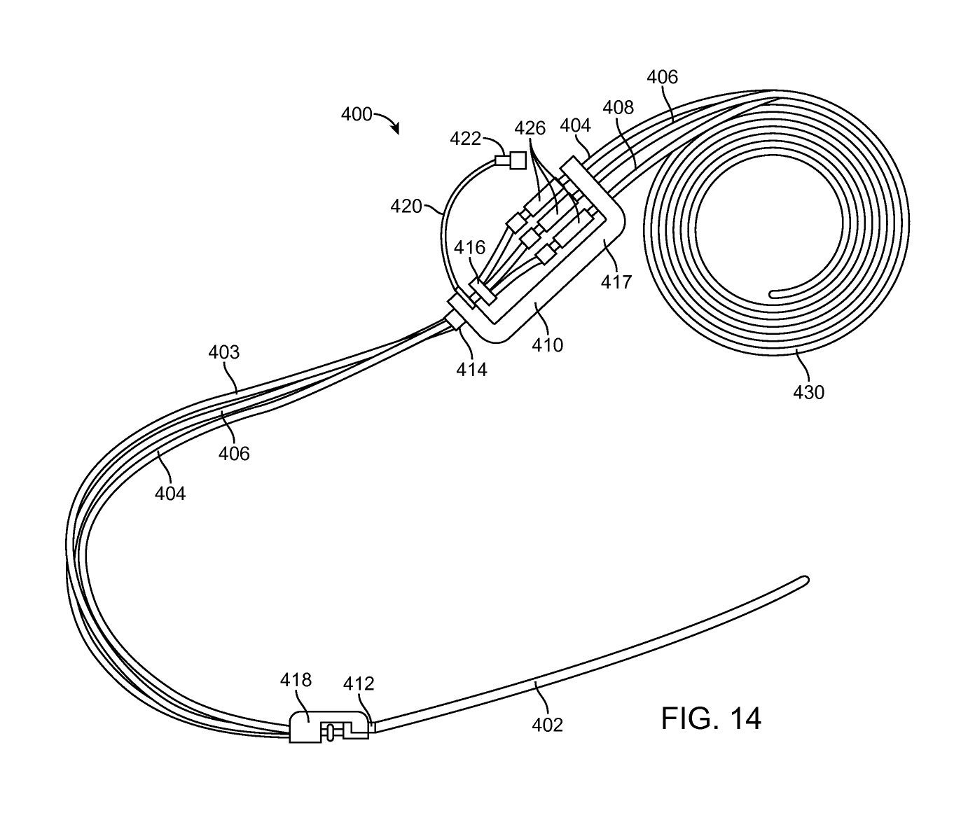

FIGS. 12 and 13 show in more detail some of the features discussed above. In FIG. 12, the issue of using a stiff guidewire that is typical state of the art, such as a Cook Amplatz Extra Stiff, is shown, where a catheter 100 is initially placed at the takeoff of an angulated artery, and a stiff guidewire 400 is advanced through the catheter and attempted to enter the distal vessel 200. When some resistance is encountered by the distal flexible portion 401 of the guidewire, the proximal guidewire 400 is too stiff to advance around the turn and the guidewire forces the distal catheter 101 to straighten and the combined guidewire and distal catheter pop out, or prolapse. As shown in FIG. 13, several smaller guidewires 10,11,12 overcome this problem by advancing individually, where each guidewire is flexible enough to advance without causing prolapsed. Once all of the parallel guidewires are in place, there is enough friction of the guidewires against the vessel wall 220 to anchor the guidewires in position and support the advancement of other sheaths or devices. This again highlights an advantage of the invention as compared to state of the art guidewires, in that when two or more guidewires are placed in a vessel, the distal guidewires have more surface area than a single guidewire, and so will have more friction against the vessel than a single guidewire, which is useful when needed to inch the guidewires when manipulating a catheter over the guidewires. Yet each guidewire has low friction when being manipulated individually during placement of each guidewire.

Referring now to FIG. 14-17, a further exemplary guidewire system 400 embodying the principles of the present invention will be described. The guidewire system 400, as best seen in FIG. 14, comprises a guidewire jacket 402, a first guidewire 404, a second guidewire 406, a third guidewire 408 and a handle 410. The guidewire jacket 402 includes a connector hub 412 at its proximal end and comprises a tubular body which is generally free along its entire length, and in particular over its distal region, from protruding structures, such as balloons and other features which would increase the diameter of the tubular body. Usually, the tubular body will have an outer diameter in the range from 0.034 inch to 0.042 inch and an inner lumen diameter in the range from 0.03 inch to 0.038 inch. Connector hub 412 may have any conventional structure, but will typically be a luer other connector type commonly used in catheters and medical devices.

The handle 410 will typically have a U-shape body or structure with a distal connector 414 at its distal end. The distal connector 414 is intended to detachably attach to the connector hub 412 on the guidewire jacket 402 and will thus typically be a luer connector which mates with the luer on the guidewire jacket. The handle 410 will optionally further include a Touhy Borst valve 416 also usually at the distal end and adjacent to the distal connector 414. The Touhy Borst valve 416 has a fluid connector line 420 and hub 422 which allows connection of the valve 416 and connector 414 to a fluid source, such as a source of radio contrast fluid. In this way, the radio contrast and other fluids may be delivered to and through the guidewire jacket 402 for various conventional purposes during the protocols described here and after.

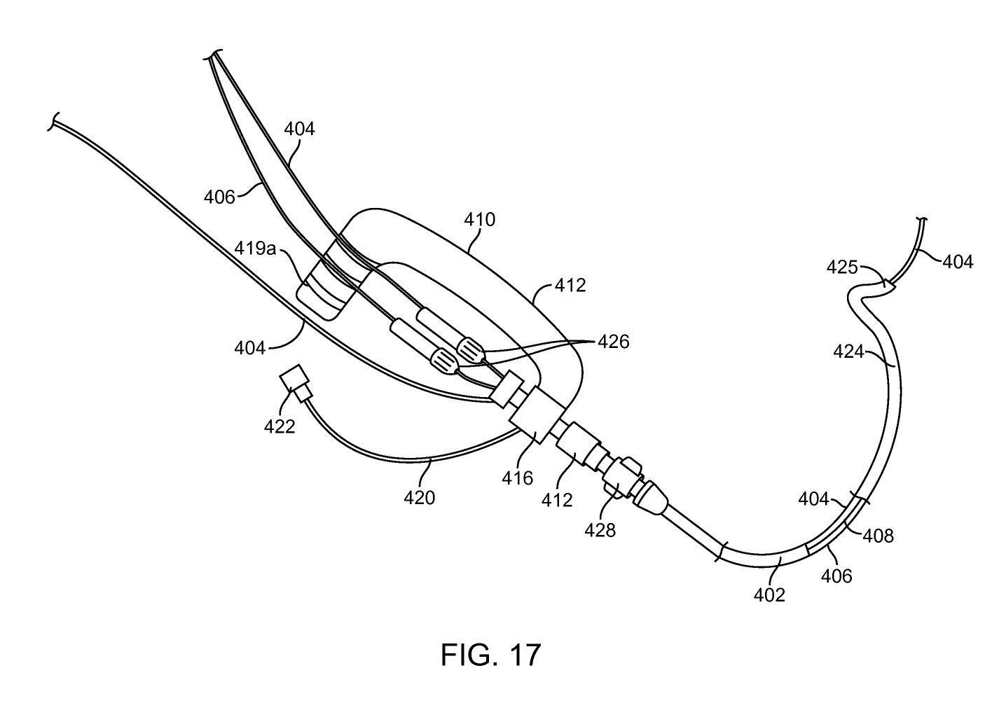

The handle 410 also has a proximal member 417 which includes a plurality of curved slots 419 (FIG. 17) which removably receive each of the guidewires 404, 406, and 408. The curved slot structures retain the guidewires in the slot by friction to allow easy detachment and replacement of the guidewires when it is desired to temporarily or permanently remove them from the handle.

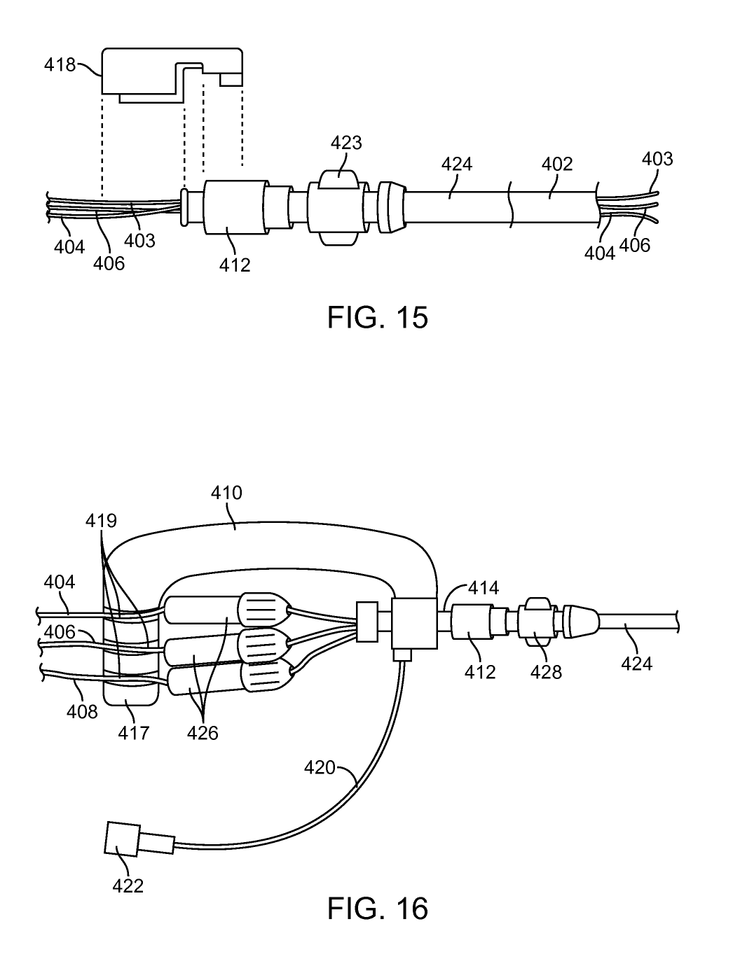

A removable retainer 418 is detachably secured to both the connector hub 412 of the guidewire jacket 402 and to distal regions of each of the guidewires 404, 406, and 408. When the retainer 414 is in place, as shown in FIG. 14, the guidewires will be immobilized relative to the guidewire jacket 402 so that they will be held in place as the guidewire is introduced to a diagnostic catheter, as described in more detail below. When the retainer 418 is detached, as shown in FIG. 15 and FIG. 17, the guidewires will be able to freely move relative to the guidewire jacket 402 and may be individually advanced through the jacket when any given guidewire is detached from the handle 410. A torquer 426 is removably attached to each of the guidewires 404, 406, and 408, to assist in manipulating the guidewires when they are detached from the handle.

As shown in FIGS. 16 and 17, the connector hub 412 of the guidewire jacket 402, may be attached to a proximal hub 428 of a diagnostic catheter 424 when the guidewire jacket 402 has been inserted into a lumen of the diagnostic catheter, as will be described in more detail below.

As shown in FIG. 17, the first guidewire 404 may be detached from slot 419a the handle 10, and the torquer 426 may be utilized to advance the guidewire through both the guidewire jacket 402 and the diagnostic catheter 424 so that the guidewire 404 emerges from distal end 425 of the diagnostic catheter. It will be appreciated that each of the guidewires 406 and 408 may be similarly detached from the handle 410 and advanced through the guidewire jacket 402 and the diagnostic catheter 424 to advance all three guidewires to a target region so that they lie in parallel and provide a continuous rail for tracking from an entry location to the target region. Once the guidewires 404, 406, and 408 are thus positioned, the diagnostic catheter 424 and the guidewire jacket 402 may be simultaneously removed over the wires, conveniently using the handle 410. The wires will usually be sufficiently long so that the portion of the wires lying outside of the patient will be longer than the length of the guidewire jacket 402 so that there will always be a portion of the guidewire accessible as the diagnostic catheter 424 and guidewire jacket 402 are being removed.

Guidewire system 400 also includes a guidewire race 430 as shown in FIG. 14. Other suitable means for managing the proximal ends of the guidewires could also be utilized.

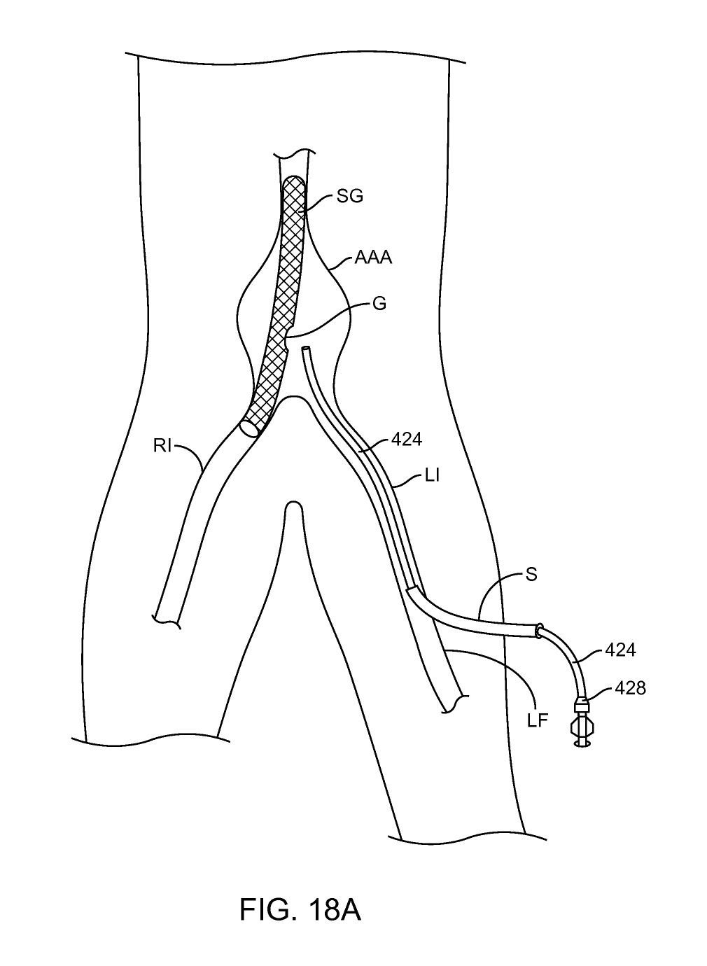

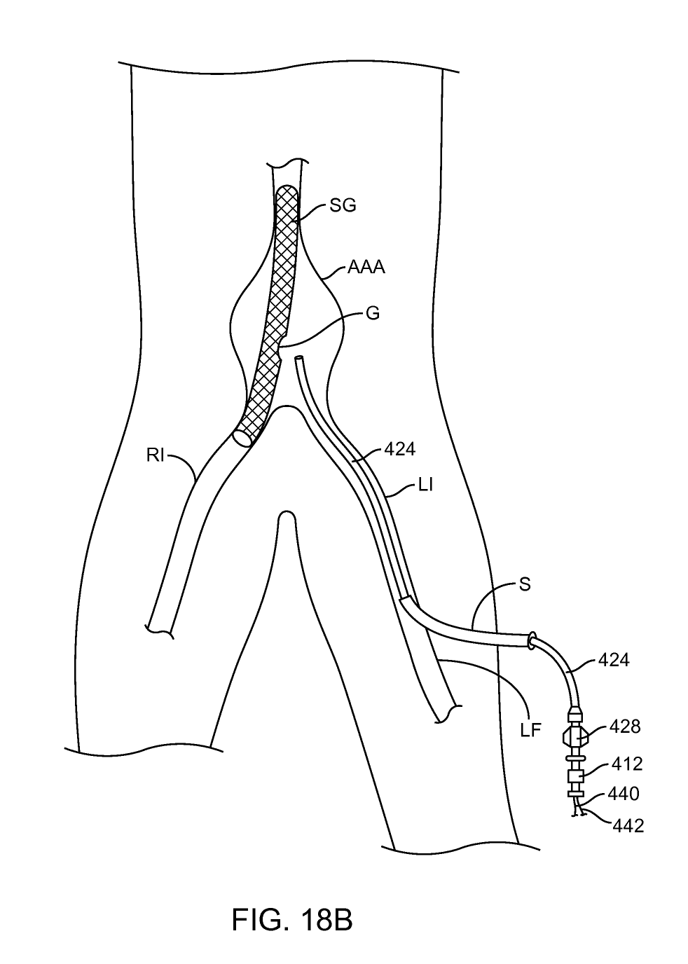

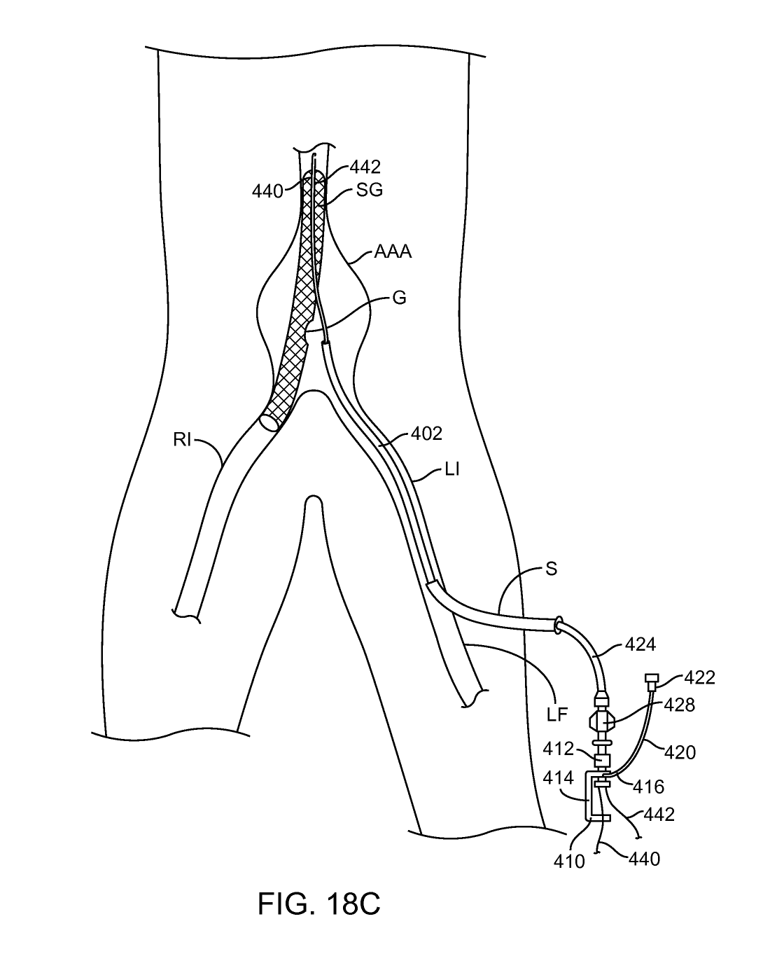

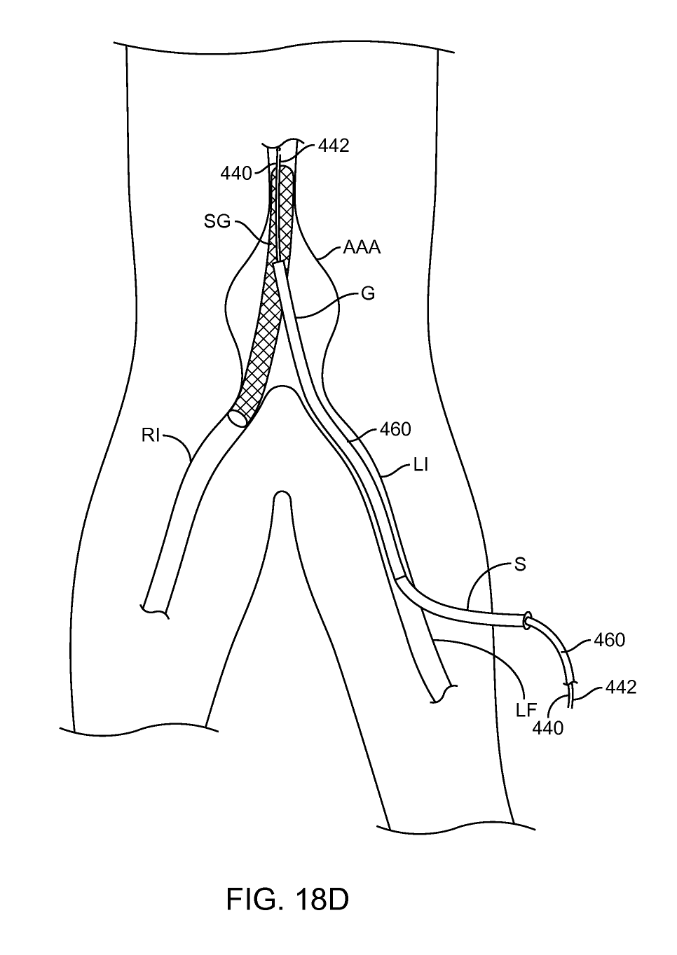

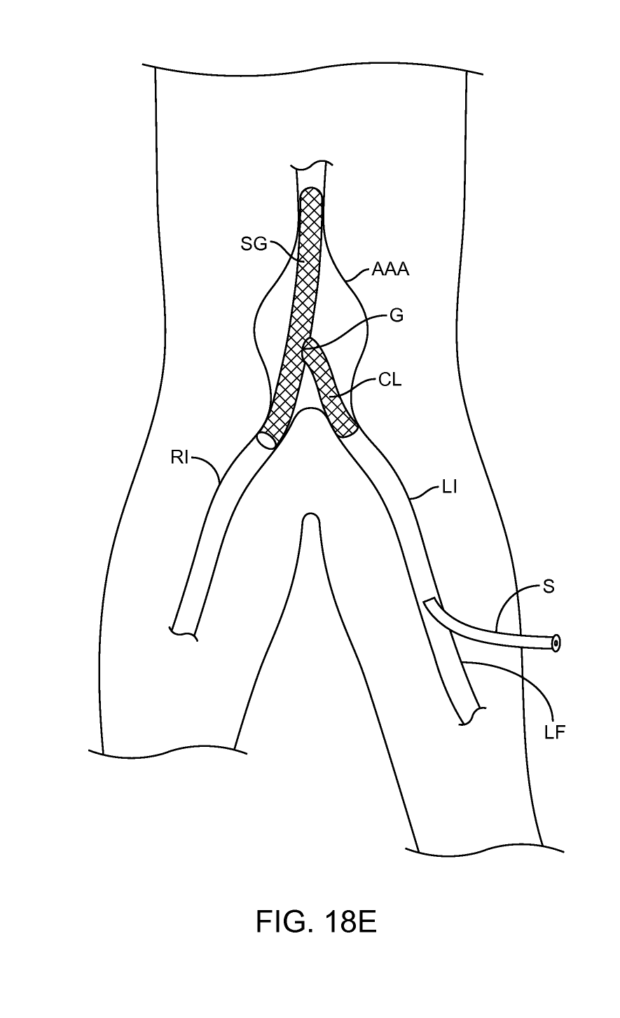

Referring now to FIGS. 18A to 18E, placement of a contralateral leg of a stent-graft assembly in an abdominal aortic aneurysms AAA will be described. As shown in FIG. 18A, the main body of a stent-graft SG has been placed between the aorta above the aneurysms AAA and the right iliac RI. An opening or gate G is present in the stent-graft and is intended to receive a short stent-graft portion referred to as a contralateral leg would will extend into the left iliac LI. Initially, a diagnostic catheter 424 is in place through a sheath S which enters the left femoral LF in a generally conventional manner.

To introduce the contralateral leg of the stent-graft, a pair of guidewires 440 and 442 (FIGS. 18B through 18D) will be placed using the guidewire placement system of the present invention. First, the guidewire jacket 402 is introduced into the lumen of the diagnostic catheter 424 and the connector hub 412 of the the guidewire jacket is connected to the proximal hub 428 of the diagnostic catheter 424, as shown in FIG. 18B. In this figure, the body of the guidewire jacket 402 is not visible since it lies entirely within the lumen of the diagnostic catheter 424. The wires 440 and 442 extend proximally from the connector hub 412, and the retainer 418 (FIG. 14) has been removed so that the wires are free to move.

The wires 404, 406, and 408 are then advanced simultaneously using the handle 410 until the distal connector 414 on the handle reaches and connects to the connector hub 412 on the guidewire jacket 402. As also shown in FIG. 18C, the wires 440 and 442 have been detached from the slots 419 (FIG. 16) on the handle 10, and the wires individually advanced until they extend through the center of the stent-graft SG and into the aorta above the abdominal arotic aneurysm AAA. Once the wires have been advanced beyond the stent-graft, radiopaque markers on at least one of the wires maybe observed so that placement of the wires can be confirmed.

Once placement of the wires 440 and 442 has been confirmed, the guidewire jacket 402 and diagnostic catheter 424 may be removed, conveniently using handle 402 to withdraw them over the wires. The wires are then available, as shown FIG. 18D, to introduce a stent delivery catheter 460 through the gate G of the stent-graft SG. The delivery catheter 460 may be any one of various conventional catheters, and would particularly release a self-expanding contralateral leg CL so that it expands into the gate G and the upper end of the left iliac LI, as shown in FIG. 18E. Once the contralateral leg has been placed, the catheter 460 and guidewire maybe removed, and other portions of the stent-graft implantation may be performed.

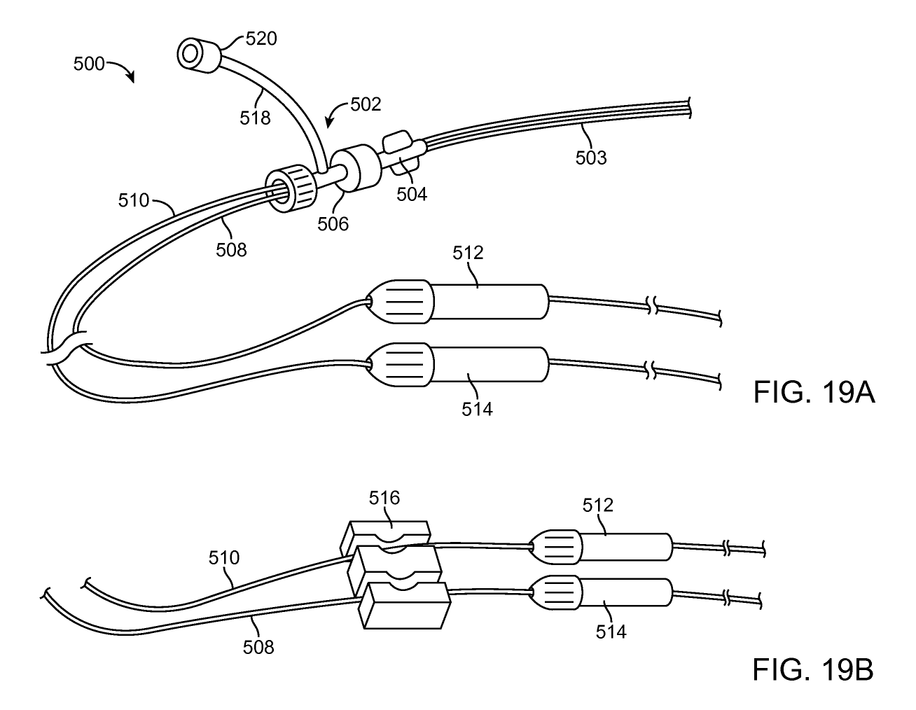

A further alternative construction of the guidewire system of the present invention is shown in FIGS. 19A and 19B. The guidewire system 500 includes a guidewire jacket 502 having a tubular body 503 with a luer connector 504 at its proximal end. A Touhy Borst connector 506 is attached to the luer connector 504 and will usually include a fluid connector or tube 518 having a connector port 520 for producing contrast or other fluids through the guidewire jacket 502. The guidewire system 500 is not intended to be used with a handle as with the previously described embodiments. For that reason, the fluid connector 518 and port 520 are provided directly on a Touhy Borst which is affixed to the luer.

First guidewire 508 and second guidewire 510 are illustrated as being partially advanced into the tubular body 503 of the guidewire jacket 502. Optionally, a guidewire retainer (not shown, but similar to retainer 418 illustrated previously) may be provided to immobilize the guidewires 508 and 510 onto the Touhy Borst connector 506 and/or luer 504. A first torquer 512 and a second torquer 514 are shown to be mounted on the first guidewire 508 and second guidewire 510, respectively, optionally, a guidewire organizer 516 may be employed, as shown in FIG. 19B. As shown in FIGS. 19A and 19B, the guidewire system 500 is ready to be advanced into and through a diagnostic or other previously positioned catheter. Once in place, the guidewires 508 and 510 may be advanced individually or simultaneously into the patient in a manner analogous to that previously described with reference to FIGS. 18A-18E.

Although specific anatomical situations are described herein, it should be clear to those versed in the art that there are many other body lumen presentations that the invention will have utility in. It should also be clear that although specific examples of the invention are disclosed, the invention is not limited to these descriptions and there are many versions of the invention that can be generated from the basic concept of using parallel guidewires of similar or different geometries, with or without being enclosed in a tube jacket, that can be encompassed and anticipated in the basic parameters of the invention.

What is claimed is:

1. A guidewire system comprising:
a guidewire jacket having a proximal end, a distal end, and a connecter hub at its proximal end; and
at least two guidewires slidably received in a lumen of the guidewire jacket, said guidewires having lengths greater than twice that of the guidewire jacket.

2. A guidewire system as in claim 1, further comprising a handle which removably receives each guidewire and has a distal connector which detachably attaches to the connector hub on the guidewire jacket.

3. A guidewire system as in claim 1, further comprising a wire organizer which removable receives the guidewires but which is not configured to attach to the hub on the guidewire jacket.

4. A guidewire system as in claim 1, wherein the guidewire jacket includes a side port on the hub, configured to provide for fluid introduction into a lumen of the jacket.

5. A guidewire system as in claim 4, wherein the hub comprises a Touhy Borst connector.

6. A guidewire system as in claim 1, wherein a distal region of the guidewire jacket consists of a tubular element which is free from exterior structure.

7. A guidewire system as in claim 1, wherein the connector hub on the guidewire jacket comprises a laer and the distal connecter on the handle comprises a Touhy Borst valve.

8. A guidewire system as in claim 1, further comprising a removable retainer which holds a distal region of each guidewire to the connector hub on the guidewire jacket.

9. A guidewire system as in claim 8, wherein the system is assembled with distal tips of the guidewires received in a proximal end of the guidewire jacket lumen, the removable retainer holding the wires in placed, and the handle spaced proximally of the retainer and connecter hub so that the guidewire jacket may be placed into a catheter, the connector hub attached to a hub on the catheter, the retainer removed, and the handle advanced to advance the guidewires through the guidewire jacket lumen.

10. A guidewire system as in claim 1, further comprising a container which holds the assembled guidewire jacket, guidewires, and handle in a sterilized condition.

11. A guidewire system as in claim 1, wherein at least one guidewire has a greater stiffness than at least one other guidewire.

12. A guidewire system as in claim 11, comprising at least three guidewires slidably received in the lumen of the guidewire jacket.

13. A guidewire system as in claim 1, further comprising torquers removably attached to each guidewire.

14. A guidewire system as in claim 1, further comprising a wire loop which receives proximal ends of the guidewires which extend proximally from the handle.

15. A guidewire system as in claim 1, comprising at least one larger guidewire, having a distal diameter in the range from 0.014 inch to 0.028 and at least one smaller guidewire having a distal diameter in the range from 0.009 inch to 0.018 inch.

16. A guidewire system as in claim 15, consisting of one larger diameter guidewire and two smaller diameter guidewires.

17. A guidewire system as in claim 15, consisting of one larger diameter guidewire and one smaller diameter guidewire with a pigtail distal end.

18. A guidewire system as in claim 1, wherein at least some of the guidewires have radiopaque markers at or near their distal ends.

19. A method for advancing a second catheter through a patient's vasculature to a target region, said method comprising:
introducing a guidewire jacket to a lumen of a first catheter which was pre-placed into the vasculature;
advancing a first guidewire from the guidewire jacket into and through the first catheter lumen to the target region;
advancing at least a second guidewire from the guidewire jacket into and through the first catheter lumen to the target region in parallel to the first guidewire;
removing the guidewire jacket and first catheter after two or more guidewires have been advanced leaving the advanced guidewires in place; and
advancing the second catheter over the advanced parallel guidewires to the target region.

20. A method as in claim 19, wherein the first catheter comprises a diagnostic catheter pre-placed into the vasculature and second catheter comprises a guiding catheter or sheath.

21. A method as in claim 19, wherein introducing a guidewire jacket comprises connecting a proximal end of the guidewire jacket to a proximal end of the first catheter.

22. A method as in claim 21, wherein distal regions of the guidewires are detachably attached to the proximal end of the guidewire jacket, further comprising detaching the guidewires from the proximal end of the guidewire jacket after the guidewire jacket has been introduced into the lumen of the first catheter and prior to the advancing guidewires to the target region.

23. A method as in claim 22, wherein a middle section of each guidewire is detachably attached to a handle or wire organizer which allows the guidewires to be simultaneously advanced through the guidewire jacket by advancing the handle or wire organizer relative to the guidewire jacket.

24. A method as in claim 23, wherein the middle sections of the guidewires to which the handle is detachably attached are located proximally of the distal ends of each guidewire at a distance which positions the distal ends of the guidewires near a distal end of the guidewire jacket and the first catheter when the handle is advanced.

25. A method as in claim 24, wherein after the handle or wire organizer has been advanced, individual guidewires are detached from the handle or wire organizer and manipulated while the other guidewire(s) remain attached to the handle.

26. A method as in claim 19, further comprising delivering contrast medium through the guidewire jacket to the target region.

27. A method as in claim 26, wherein the contrast medium is delivered through a Touhy Borst valve on a proximal end of the guidewire jacket.

28. A method as in claim 19, wherein the first catheter comprises a diagnostic catheter placed from an aortic arch to a carotid artery.

29. A method as in claim 28, wherein the second catheter delivers a stent to the carotid artery.

30. A method as in claim 29, wherein the first catheter comprises a diagnostic catheter disposed in a splenic artery and the target region is an aneurysm.

31. A method as in claim 30, wherein the second catheter delivers a stent-graft to the aneurysm.

32. A method as in claim 19, wherein the first catheter comprises a diagnostic catheter placed from a femoral artery to an aorta.

33. A method as in claim 32, wherein the target region comprises a contralateral gate in a stent-graft pre-placed in an aneurysm in the abdominal aorta.

34. A method as in claim 33, wherein a distal end of the first catheter is placed near but not through the contralateral gate, the first wire is advanced through the gate, and the first catheter is the advanced over the first wire through the gate and into the stent-graft.

35. A method as in claim 34, wherein the first and second guidewires are advanced from the jacket through the stent-graft so that markers present on at least one of the guidewires are visible distal to the stent jacket aid removing the first catheter and guidewire jacket from over the first and second guidewires.

36. A method as in claim 35, wherein the second catheter delivers a contralateral stent graft to the contralateral gate.

37. A method as in claim 19, wherein the first catheter is positioned through a femoral artery, over the aortic bifurcation to a contralateral iliac artery.
